(19) United States
(12) Patent Application Publication
(10) Pub. No.: US 2007/0067181 A1
(43) Pub. Date: Mar. 22, 2007

(54) System and method for intelligence building in an expert system

(75) Inventors: Richard D. Dettinger, Rochester, MN (US); Judy I. Djugash, Rochester, MN (US); Daniel P. Kolz, Rochester, MN (US)

Correspondence Address: IBM CORPORATION, INTELLECTUAL PROPERTY LAW;DEPT 917, BLDG. 006-1, 3605 HIGHWAY 52 NORTH, ROCHESTER, MN, 55901-7829, US

(73) Assignee: INTERNATIONAL BUSINESS MACHINES CORPORATION, ARMONK, NY (US)

(21) Appl. No.: 11/232,766

(22) Filed: Sep. 22, 2005

(51) Int. Cl. G06Q 10/00; A61B 5/00

(52) U.S. Cl. 705/2; 600/300

(57) ABSTRACT

A method, system and article of manufacture for intelligence building in expert systems and, more particularly, for evaluating treatment decisions overriding recommended treatments that are generated by expert systems. One embodiment provides a method of evaluating treatment decisions, comprising receiving a specification of a decided treatment for a patient having a given disease. The decided treatment differs from a recommended treatment identified by an expert system in response to analysis of symptomatic data corresponding to the patient. The patient is monitored subsequent to being treated according to the decided treatment. The decided treatment is evaluated on the basis of data captured by the monitoring. On the basis of the evaluation, feedback with respect to the decided treatment and the recommended treatment is generated.

### BACKGROUND OF THE INVENTION

1. Field of the Invention

The present invention generally relates to expert systems and more particularly to mechanisms for intelligence building in expert systems.

2. Description of the Related Art

The creation of increasingly powerful computer (or computing) systems and a continuously improved information technology (IT) infrastructure contribute to a progressive automation of today's tasks and processes. As a result, use and development of computer-based expert systems, such as case-based reasoning (CBR) systems, continuously progress in a variety of different domains.

A CBR system generally refers to a computer system that identifies a solution to a current problem by examining descriptions of similar, previously encountered problems and their associated solutions. The current problem is matched with one or more similar previously encountered problems and the associated solutions of the matching previously encountered problems are used to suggest a solution to the current problem. In other words, in response to receipt of a description of a current problem, a conventional CBR system retrieves the closest matching cases from a case database using a search engine. Thereby, a user can be prompted iteratively for additional descriptive information until the retrieved case or cases identified by the search engine are sufficiently similar to the current problem to be considered as possible solutions. If a solution to a problem which is not pre-stored in the case database is subsequently validated, the validated solution can be entered into the case database and utilized to solve future problems.

Recent developments of CBR systems in healthcare and life sciences focus on improvements of medical expert systems which are suitable to provide treatment recommendations to doctors. Such expert systems allow doctors to consult historical data on patients and symptoms to treat a current case easily and securely.

However, there may be cases where different possible treatments could apply to a given set of symptoms or cases where the doctor intends to provide another treatment than the one which is recommended by an underlying expert system. In such cases, the doctor is allowed to override the system's treatment recommendation. For instance, in some cases the doctor may choose to resolve a specific situation presented by a given case on the basis of personal experience and expertise. In other words, the doctor may decide to perform a particular treatment assuming that the recommended treatment would be less effective in the specific situation. If the doctor's decision is correct, overriding the recommended treatment would be advantageous for the patient. But, of course, it may be that the treatment decided on by the doctor is incorrect and/or not as efficient as would be the treatment that was recommended by the expert system. In any case the consequences of the doctor's decision are not preserved for future intelligence building in the expert system.

Therefore, there is a need for improved mechanisms for intelligence building in expert systems.

### SUMMARY OF THE INVENTION

The present invention generally is directed to a method, system and article of manufacture for intelligence building in expert systems and, more particularly, for evaluating treatment decisions overriding recommended treatments that are generated by expert systems.

One embodiment provides a computer-implemented method of evaluating treatment decisions, comprising receiving a specification of a decided treatment for a patient having a given disease. The decided treatment differs from a recommended treatment identified by an expert system in response to analysis of symptomatic data corresponding to the patient. The method further comprises creating an outcome test specification defining at least one check point with respect to the given disease which is suitable for evaluation of a treatment result obtained with the decided treatment. On the basis of the outcome test specification, a database watcher is created. The database watcher is configured to analyze data which is subsequently provided for the patient with respect to the at least one check point to monitor progress of the given disease. The database watcher is executed to determine the treatment result on the basis of the analyzed data, and a medical institution is notified of the determined treatment result.

Another embodiment provides another computer-implemented method of evaluating treatment decisions, comprising receiving a specification of a decided treatment for a patient having a given disease, the decided treatment differing from a recommended treatment identified by an expert system in response to analysis of symptomatic data corresponding to the patient. The method further comprises monitoring the patient subsequent to being treated according to the decided treatment. The decided treatment is evaluated on the basis of data captured by the monitoring. On the basis of the evaluation, feedback with respect to the decided treatment and the recommended treatment is generated.

Still another embodiment provides a system, comprising an expert system and an intelligence unit for evaluating treatment decisions. The expert system is configured to determine a recommended treatment in response to analysis of symptomatic data corresponding to a patient having a given disease. The intelligence unit is configured to: (i) receive a specification of a decided treatment for the patient, the decided treatment differing from the recommended treatment; (ii) create an outcome test specification defining at least one check point with respect to the given disease which is suitable for evaluation of a treatment result obtained with the decided treatment; (iii) create, on the basis of the outcome test specification, a database watcher configured to analyze data which is subsequently provided for the patient with respect to the at least one check point to monitor progress of the given disease; (iv) execute the database watcher to determine the treatment result on the basis of the analyzed data; (v) evaluate the decided treatment on the basis of the treatment result; and (vi) generate, on the basis of the evaluation, feedback with respect to the decided treatment and the recommended treatment.

### BRIEF DESCRIPTION OF THE DRAWINGS

So that the manner in which the above recited features, advantages and objects of the present invention are attained and can be understood in detail, a more particular description of the invention, briefly summarized above, may be had by reference to the embodiments thereof which are illustrated in the appended drawings.

It is to be noted, however, that the appended drawings illustrate only typical embodiments of this invention and are therefore not to be considered limiting of its scope, for the invention may admit to other equally effective embodiments.

FIG. 1 is one embodiment of a computer system utilized in accordance with the invention;

FIG. 2 is a relational view of software components of one embodiment of the invention;

FIGS. 3A-B are flow charts illustrating a method of evaluating treatment decisions in one embodiment; and

FIG. 4 is a flow chart illustrating a method of executing a database watcher to determine a treatment result in one embodiment.

### DETAILED DESCRIPTION OF THE PREFERRED EMBODIMENTS

Introduction

The present invention is generally directed to a method, system and article of manufacture for intelligence building in expert systems and, more particularly, for evaluating treatment decisions overriding recommended treatments that are generated by expert systems. In general, an expert system for generation of treatment recommendations is configured to determine a recommended treatment for a patient having a given disease. The recommended treatment is generated in response to analysis of symptomatic data corresponding to the patient.

In one embodiment, symptomatic data for a given patient is created on the basis of symptoms exhibited by a given patient. The symptomatic data is entered into the expert system and the doctor, or other user, requests the expert system to identify a probable diagnosis of a disease corresponding to the symptomatic data and to determine a recommended treatment that is suitable to treat the disease. The expert system analyzes the symptomatic data and issues a recommended treatment for the diagnosed disease to the doctor. Upon receipt of the recommended treatment, the doctor may accept the recommended treatment or decide to perform another treatment on the given patient.

In one embodiment, the doctor decides to perform a treatment that differs from the recommended treatment. In this case, an underlying intelligence unit records the decided treatment of the doctor and any deviations from the recommended treatment of the expert system. The intelligence unit further creates an outcome test specification having one or more check points that are suitable to evaluate progress of the diagnosed disease. By way of example, these checkpoints include questions about future diagnosis and tests that are suitable to determine the progress of the diagnosed disease.

According to one aspect, the checkpoints are created and stored by a manager agent which can be implemented by the intelligence unit. The manager agent further stores data which is obtained by monitoring the patient subsequent to being treated according to the decided treatment. The decided treatment may thus be evaluated on the basis of the data that is captured by the monitoring. On the basis of the evaluation, feedback with respect to the decided treatment and the recommended treatment is generated and, for instance, reported to the doctor, the expert system, a quality control system, and/or a medical institution. Thus, the impact of the doctor's decision on the patient can be preserved for future intelligence building in the expert system.

### PREFERRED EMBODIMENTS

In the following, reference is made to embodiments of the invention. However, it should be understood that the invention is not limited to specific described embodiments. Instead, any combination of the following features and elements, whether related to different embodiments or not, is contemplated to implement and practice the invention. Furthermore, in various embodiments the invention provides numerous advantages over the prior art. However, although embodiments of the invention may achieve advantages over other possible solutions and/or over the prior art, whether or not a particular advantage is achieved by a given embodiment is not limiting of the invention. Thus, the following aspects, features, embodiments and advantages are merely illustrative and, unless explicitly present, are not considered elements or limitations of the appended claims.

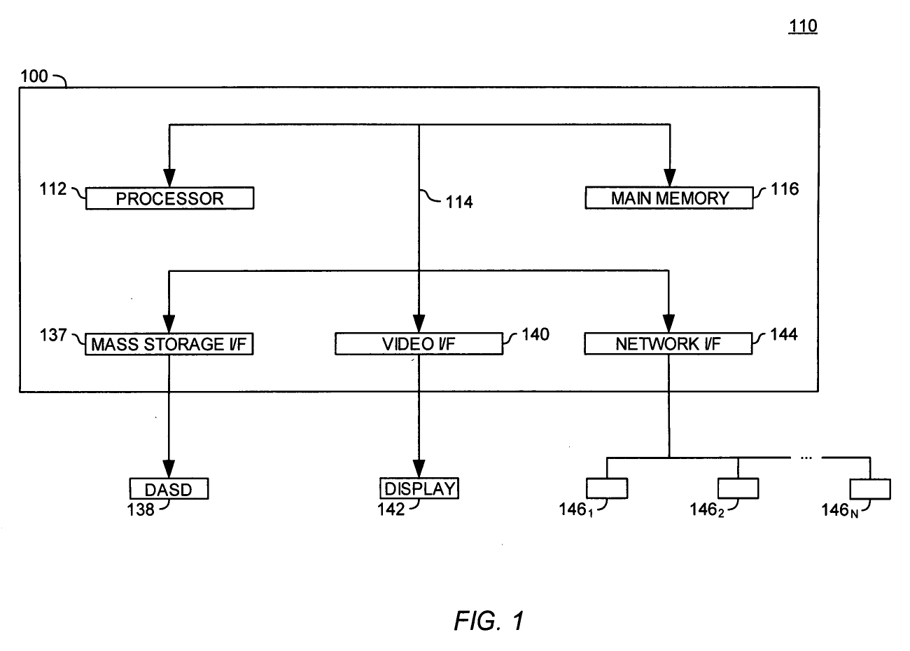

One embodiment of the invention is implemented as a program product for use with a computer system such as, for example, computer system 110 shown in FIG. 1 and described below. The program(s) of the program product defines functions of the embodiments (including the methods described herein) and can be contained on a variety of computer-readable media. Illustrative computer-readable media include, but are not limited to: (i) information permanently stored on non-writable storage media (e.g., read-only memory devices within a computer such as CD-ROM disks readable by a CD-ROM drive); (ii) alterable information stored on writable storage media (e.g., floppy disks within a diskette drive or hard-disk drive); or (iii) information conveyed to a computer by a communications medium, such as through a computer or telephone network, including wireless communications. The latter embodiment specifically includes information to/from the Internet and other networks. Such computer-readable media, when carrying computer-readable instructions that direct the functions of the present invention, represent embodiments of the present invention.

In general, the routines executed to implement the embodiments of the invention, may be part of an operating system or a specific application, component, program, module, object, or sequence of instructions. The software of the present invention typically is comprised of a multitude of instructions that will be translated by the native computer into a machine-readable format and hence executable instructions. Also, programs are comprised of variables and data structures that either reside locally to the program or are found in memory or on storage devices. In addition, various programs described hereinafter may be identified based upon the application for which they are implemented in a specific embodiment of the invention. However, it should be appreciated that any particular nomenclature that follows is used merely for convenience, and thus the invention should not be limited to use solely in any specific application identified and/or implied by such nomenclature.

### An Exemplary Computing Environment

FIG. 1 shows a computer 100 (which is part of a computer system 110) that becomes a special-purpose computer according to an embodiment of the invention when configured with the features and functionality described herein. The computer 100 may represent any type of computer, computer system or other programmable electronic device, including a client computer, a server computer, a portable computer, a personal digital assistant (PDA), an embedded controller, a PC-based server, a minicomputer, a midrange computer, a mainframe computer, and other computers adapted to support the methods, apparatus, and article of manufacture of the invention. Illustratively, the computer 100 is part of a networked system 110. In this regard, the invention may be practiced in a distributed computing environment in which tasks are performed by remote processing devices that are linked through a communications network. In a distributed computing environment, program modules may be located in both local and remote memory storage devices. In another embodiment, the computer 100 is a standalone device. For purposes of construing the claims, the term “computer” shall mean any computerized device having at least one processor. The computer may be a standalone device or part of a network in which case the computer may be coupled by communication means (e.g., a local area network or a wide area network) to another device (i.e., another computer).

In any case, it is understood that FIG. 1 is merely one configuration for a computer system. Embodiments of the invention can apply to any comparable configuration, regardless of whether the computer 100 is a complicated multi-user apparatus, a single-user workstation, or a network appliance that does not have non-volatile storage of its own.

The computer 100 could include a number of operators and peripheral systems as shown, for example, by a mass storage interface 137 operably connected to a storage device 138, by a video interface 140 operably connected to a display 142, and by a network interface 144 operably connected to the plurality of networked devices 146 (which may be representative of the Internet) via a suitable network. Although storage 138 is shown as a single unit, it could be any combination of fixed and/or removable storage devices, such as fixed disc drives, floppy disc drives, tape drives, removable memory cards, or optical storage. The display 142 may be any video output device for outputting viewable information.

Computer 100 is shown comprising at least one processor 112, which obtains instructions and data via a bus 114 from a main memory 116. The processor 112 could be any processor adapted to support the methods of the invention. In particular, the computer processor 112 is selected to support the features of the present invention. Illustratively, the processor is a PowerPC® processor available from International Business Machines Corporation of Armonk, N.Y.

The main memory 116 is any memory sufficiently large to hold the necessary programs and data structures. Main memory 116 could be one or a combination of memory devices, including Random Access Memory, nonvolatile or backup memory, (e.g., programmable or Flash memories, read-only memories, etc.). In addition, memory 116 may be considered to include memory physically located elsewhere in the computer system 110, for example, any storage capacity used as virtual memory or stored on a mass storage device (e.g., direct access storage device 138) or on another computer coupled to the computer 100 via bus 114. Thus, main memory 116 and storage device 138 could be part of one virtual address space spanning multiple primary and secondary storage devices.

### An Exemplary System for Evaluating Treatment Decisions

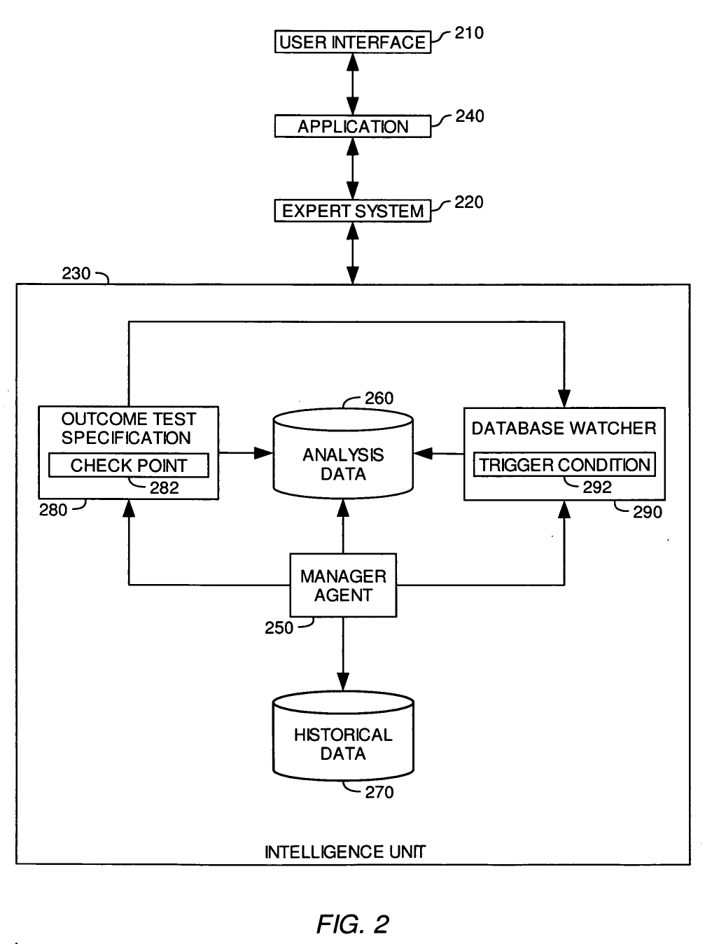

Referring now to FIG. 2, a relational view of software components in one embodiment is illustrated. The software components illustratively include a user interface 210, an expert system 220, an intelligence unit 230 and one or more applications 240 (only one application is illustrated for simplicity). The intelligence unit 230 illustratively includes a manager agent 250, a database 260 for storing analysis data and a database 270 for storing historical data.

The user interface 210 can be any suitable user interface that is configured to allow user interaction with the application 240. In one embodiment, the user interface 210 is a graphical user interface that is configured to input symptomatic data for patients. The symptomatic data for a patient having a given disease corresponds to symptoms exhibited by the patient. For instance, during a patient's visit with a doctor in a medical institution, the patient describes several symptoms. Symptomatic data may also be derived from various tests including lab tests, physical examinations, magnetic resonance imaging, CAT scans, etc. In any case, the doctor may input the symptomatic data for the patient using the user interface 210.

According to one aspect, the application 240 (and more generally, any requesting entity including, at the highest level, users) submits the symptomatic data to the expert system 220. The expert system 220 then determines a recommended treatment for the patient in response to analysis of the symptomatic data. To this end, the expert system 220 initially establishes a diagnosis of the patient's condition. In one embodiment, the expert system 220 may unambiguously identify a single disease. If, however, an unambiguous diagnosis is not possible with the data input thus far, the expert system 220 may request additional symptomatic data.

For instance, assume a patient having a body temperature greater than 99.5° F. who complains about body aches. However, these two symptoms occur in patients having influenza as well as strep throat. Assume now that the expert system 220 requests further information and that additional symptomatic data for the patient specifies sore throat symptoms, which is a typical symptom of strep throat, but not of influenza. Thus, strep throat can unambiguously be diagnosed. In other cases, an unambiguous diagnosis may not be necessary because the treatment for the two or more possible diagnoses determined on the basis of the available data may be the same (e.g., some regimen of antibiotics).

According to one aspect, the expert system 220 accesses a suitable knowledge database to identify a recommended treatment for the patient on the basis of the established diagnosis. However, it should be noted that any suitable technique for determining the recommended treatment on the basis of the established diagnosis is broadly contemplated and, therefore, not described in more detail.

In one embodiment, the manger agent 250 stores the recommended treatment and the symptomatic data in the database 260. The database 260 is illustratively shown as part of the intelligence unit 230. However, it should be noted that the database 260 can also be provided as a separate component. Furthermore, any other suitable component can be used to store the symptomatic data and the recommended treatment in the database 260, e.g., the application 240. All such different implementations are broadly contemplated.

The database 260 is configured to store analysis data for a multiplicity of patients. The analysis data of each patient associates symptomatic data of the patient with data that uniquely identifies the patient. According to one aspect, the analysis data includes personal information for each patient, such as name, address, date of birth and phone number. Furthermore, the analysis data of each patient includes an indication of a diagnosed disease and a description of a recommended treatment for the patient.

In one embodiment, the recommended treatment and the established diagnosis for the given patient are issued from the expert system 220 to the application 240. The application 240 provides the recommended treatment and the established diagnosis to the doctor, e.g., via the user interface 210. Thus, the doctor may evaluate the established diagnosis on the basis of his/her own experience and expertise and decide on a suitable treatment for the given patient. More specifically, the doctor may choose to perform the recommended treatment or to override the recommended treatment with a decided treatment. In other words, the doctor may decide to perform a particular decided treatment for the patient assuming that the recommended treatment would be less effective in the specific situation.

If the doctor decides to perform the recommended treatment, a corresponding data record is created in the database 270. The database 270 is illustratively shown as part of the intelligence unit 230. However, it should be noted that the database 270 can also be provided as a separate component. Accordingly, any possible different implementation is broadly contemplated. In one embodiment, the database 270 is configured to store historical data, i.e., data that is related to previous cases handled by the medical institution. Each case is described by one or more data records in the database 270 which include at least: (i) analysis data for a patient, (ii) a unique identification of a treating doctor, and (iii) an indication that the doctor has decided to perform the recommended treatment.

If, however, the doctor chooses to override the recommended treatment with a decided treatment, all deviations of the decided treatment from the recommended treatment are determined and stored in the analysis data database 260. Furthermore, the manager agent 250 may audit the doctor in order to determine reasons for the overriding of the recommended treatment. The provided reasons can then be stored together with the determined deviations. Moreover, the manager agent 250 can be configured to notify the medical institution of the decided treatment. This may enable the medical institution to accept or refuse the decided treatment. In other words, the medical institution may prevent the doctor from overriding the recommended treatment. This may be required in cases where a high degree of liability faces the institution if the doctor's reasons for overriding the recommended treatment are not well-founded. This may also be desirable in cases where the doctor disagrees with the system more often than the average and he/she has a success rate in similar cases which is below average. In one embodiment, such intelligence can be captured and built into the expert system 220 and provided to the medical institution when the doctor decides to override the recommended treatment.

In one embodiment, the manager agent 250 is configured to create an outcome test specification 280 for the decided treatment. The outcome test specification 280 defines at least one check point 282 with respect to the diagnosed disease which is suitable for evaluation of a treatment result obtained with the decided treatment. In a particular embodiment, the at least one check point 282 identifies one of a diagnosis and a test to be performed on the patient to provide feedback with respect to the progress of the given disease. For instance, in the given example the at least one check point must be suitable to monitor progress of the strep throat. With respect to the above described symptoms, progress of strep throat can be determined by measuring the body temperature of the patient. If the body temperature decreases, the decided treatment is effective. If the temperature does not decrease, the decided treatment is ineffective. Accordingly, the body temperature can be selected as check point for a patient diagnosed with strep throat. The check point may also specify a particular time at which a test (e.g., taking the patient's temperature) is performed. Furthermore, the check point may specify a diagnosis to be performed by the doctor. For instance, the check point may specify that the doctor needs to reexamine the patient at a given point of time to determine whether an amelioration of specific symptoms, which can not be evaluated by particular tests and measurements, has occurred. By way of example, the doctor may need to look at the patient's throat to determine visually whether the inflammation caused by the strep throat is retrogressive.

It should be noted that the outcome test specification 280 can generally be created to monitor the patient's state of health subsequent to being treated according to the decided treatment. For instance, assume that in the given example it is known that the decided treatment may have side-effects on the patient's cardiac function. In this case, one or more other check points can be created to monitor the patient's cardiac function. Thus, if the cardiac function is affected by the decided treatment, the doctor or the medical institution can be alerted. Or, assume that in previous cases the decided treatment of the given example rarely affected (for unknown reasons) a patient's ability to see, but that elevated intraocular pressure values are an indication of impending reaction. Accordingly, one or more check points can be created in order to monitor the patient's intraocular pressure. Thus, if evaluated intraocular pressure values are determined for the patient, the doctor or the medical institution can also be alerted.

In one embodiment, the outcome test specification 280 is created by the manager agent 250 using a suitable knowledge database and/or the expert system 220. More specifically, the suitable knowledge database may store a corresponding outcome test specification for each possible diagnosis which can be established by the expert system 220. Furthermore, the outcome test specification 280 can be output to the doctor for review and confirmation. Thus, the doctor may modify or add check points to the outcome test specification 280 as required. Alternatively, for each specific case a particular outcome test specification can be manually created by the doctor using the user interface 210. All such implementations, and variations thereon, are broadly contemplated.

On the basis of the outcome test specification 280, the manager agent 250 creates a database watcher 290, according to one embodiment of the invention. The database watcher 290 is configured to analyze data which is subsequently provided for the patient with respect to the at least one check point 282 to monitor progress of the diagnosed disease. In one embodiment, the database watcher 290 is associated with a predefined execution duration and is executed to determine a treatment result on the basis of the analyzed data. The predefined execution duration can be determined automatically on the basis of the diagnosed disease or interactively by the doctor. For instance, in the given example the diagnosed strep throat should be healed in less than one week with the recommended treatment. Thus, the execution duration for a corresponding database watcher can be less than one week, e.g., 4 days. In other words, it can be assumed that the decided treatment is ineffective or not effective enough if no improvement in the patient's condition is detected within 4 days. Accordingly, the database watcher should determine a treatment result at least after the 4 days so that the decided treatment can be changed or adjusted if no improvement is detected.

In one embodiment, the outcome test specification 280 can be updated during the predefined execution duration. For instance, assume that the patient who is treated for strep throat makes an appointment with the doctor for another routine matter, such as a cold. Assume now that the doctor determines at the patient's visit that the patient has a simple cold, which does not require a particular treatment, but that the patient's glucose levels are too low. Accordingly, a check point can be added with respect to subsequent examination of glucose levels to identify problems with the ongoing strep throat treatment.

In one embodiment, the database watcher 290 includes at least one trigger condition 292 on a selected check point of the at least one check point 282 in order to trigger determination of the treatment result. According to one aspect, the treatment result is only determined if the trigger condition is satisfied or if the execution duration of the database watcher 290 is expired. Exemplary trigger conditions include incoming data representing a particular test value or diagnosis, data representing the death of the patient or the complete recovery of the patient, any data indicative of a sudden improvement or decline in the patient's condition or any other appropriate condition.

On the basis of the treatment result, the decided treatment can be evaluated. For instance, in the given example where a check point has been defined for the body temperature, the trigger condition may require that the body temperature exceeds a predetermined critical level, such as 103° F., to trigger determination of the treatment result. Accordingly, if the body temperature exceeds the predetermined critical level after the decided treatment, the database watcher 290 determines the treatment result and notifies the manager agent 250.

On the basis of the treatment result, the decided treatment can be evaluated. On the basis of the evaluation, feedback with respect to the decided treatment and the recommended treatment can be generated. In one embodiment, the feedback is reported to at least one of the doctor, the expert system 220, a quality control system, or a medical institution. Thus, according to one aspect the impact of the doctor's decision can be preserved for future intelligence building in the expert system 220.

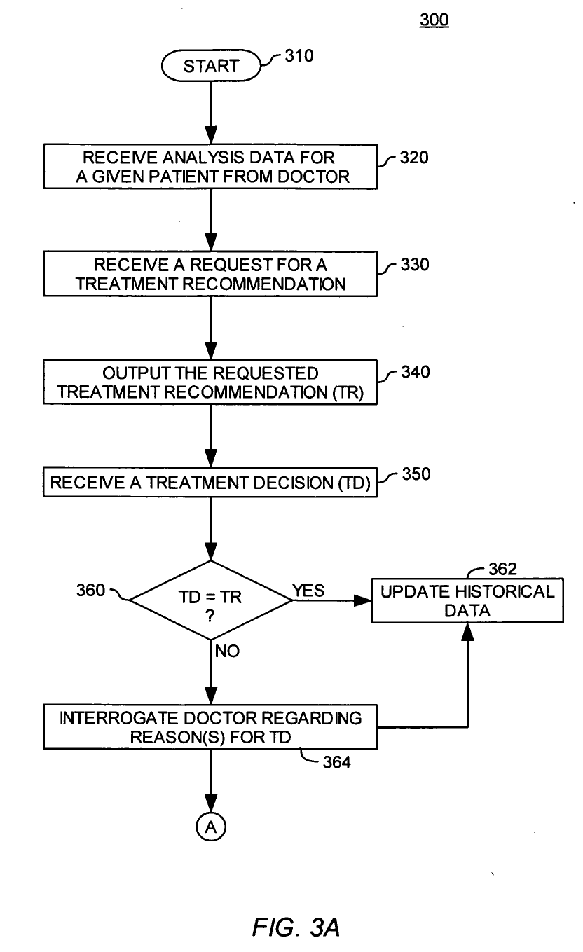

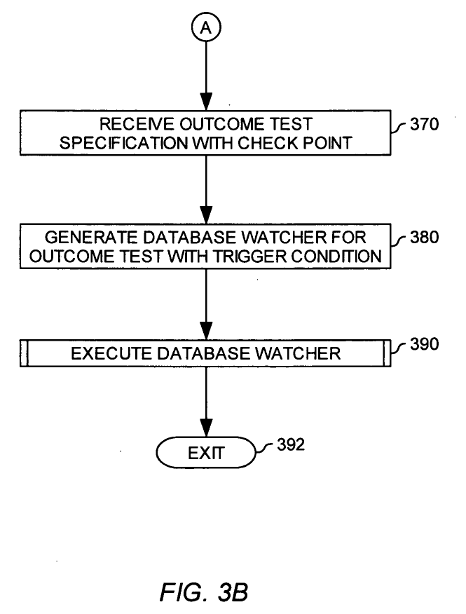

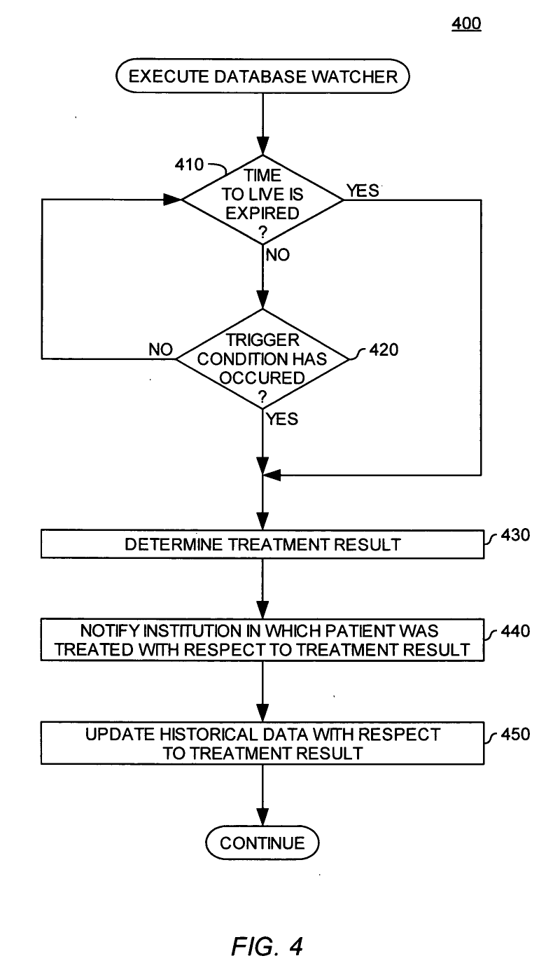

An exemplary method for evaluating treatment decisions is described in more detail below with reference to FIGS. 3-4.

### Evaluating Treatment Decisions

Referring now to FIGS. 3A-B, one embodiment of a method 300 for evaluating treatment decisions is illustrated. In one embodiment, at least part of the steps of the method 300 are performed by the expert system 220 of FIG. 2 and/or the intelligence unit 230 of FIG. 2. Furthermore, at least several steps of the method 300 can be performed on the basis of user input received via the user interface 210 of FIG. 2. Method 300 starts at step 310.

At step 320, analysis data for a given patient having a given disease is received. For instance, assume that the given patient complains about strong headaches and impaired vision. For an in-depth analysis of the patient's state of health, the doctor requests a computer tomography of the patient's head. On the basis of the described symptoms and the results of the computer tomography, the doctor creates the symptomatic data for the given patient. The doctor further associates the symptomatic data with personal information of the given patient in order to create a medical record, i.e., the analysis data for the patient.

At step 330, a request for determination of a recommended treatment for the given patient is received. At step 340, the symptomatic data is analyzed and the recommended treatment is determined. As was noted above, determining the recommended treatment may include interactively establishing an initial diagnosis of the patient's disease.

The determined recommended treatment is output to the doctor. In one embodiment, the established diagnosis is also output to the doctor. Thus, the doctor can verify whether the established diagnosis corresponds to the diagnosis that he/she establishes using his/her own experience and expertise. Assume now that in the given example the computer tomography of the patient's head has revealed a malignant brain tumor and that, accordingly, the diagnosed disease is cancer. Assume further that the recommended treatment suggests prescribing drug X to the given patient. The recommended treatment may further comprise additional information concerning use of drug X for treatment of a brain tumor. For instance, the recommended treatment may comprise an indication that for 55% of previous patients in similar life situations with similar biochemistry drug X has lead to a significant reduction of the brain tumor of up to 60%. Assume now that the recommended treatment further suggests prescribing drug Z because drug X and drug Z together were very efficient for 65% of previous patients.

At step 350, a treatment decision is received. The treatment decision includes specification of a decided treatment for the given patient. The decided treatment may be the recommended treatment or a different treatment which has been chosen by the doctor.

At step 360, it is determined whether the decided treatment is the recommended treatment. If the decided treatment is the recommended treatment, the method 300 proceeds with step 362.

At step 362, a corresponding database (e.g., historical data database 270 of FIG. 2) is updated as described above. More specifically, at step 362 a historical data record is created which indicates that the doctor has chosen the recommended treatment. The historical data record is stored in the corresponding database.

Returning to step 360, if the doctor has selected a decided treatment that differs from the recommended treatment, then at step 364, the doctor is audited. More specifically, the doctor is interrogated by the intelligence unit to determine reasons for overriding the recommended treatment. Assume now that in the given example the doctor decides to use drug Y instead of using drugs X and Z. Assume further that the doctors reasons are that (i) drugs X and Z together are too strong for the given patient whose state of health has already been weakened by the brain tumor; and (ii) drug X may have a strong side effect on the patient's vision, which has already been appreciably affected by the brain tumor.

In one embodiment, a historical data record is created using the determined reasons and the received analysis data. The historical data record can be used to update the corresponding database according to step 362. However, as was noted above the determined reasons can alternatively be stored together with the analysis data in one embodiment. Accordingly, the determined reasons may only be analyzed after determination of a treatment result for the given patient. Thus, a more meaningful analysis of the decided treatment and the determined reasons can be performed.

At step 370, an outcome test specification (e.g., outcome test specification 280 of FIG. 2) is created for the given patient with respect to the diagnosed cancer. In one embodiment, the outcome test specification is created on the basis of the symptomatic data for the given patient. As was noted above, the outcome test specification includes one or more check points (e.g., check point 282 of FIG. 2) which allow evaluation of the progress of the diagnosed cancer. In the given example, progress of the brain cancer can be determined by measuring the size of the tumor. If the size of the tumor decreases, the decided treatment using drug Y may be effective. If the size increases, the decided treatment is ineffective. Accordingly, the size of the tumor is selected as check point.

At step 380, a database watcher (e.g., database watcher 290 of FIG. 2) is created with respect to the outcome test specification. As was noted above, the database watcher includes one or more trigger conditions (e.g., trigger condition 292 of FIG. 2), each being created for a corresponding check point of the created outcome test specification. Assume now that the tumor size is 2 cm when the given patient is treated with drug Y. In this case, the trigger condition may require that the tumor size becomes twice the starting size to trigger determination of the treatment result. For instance, it is assumed that the patient's state of health becomes critical if the tumor size exceeds 4 cm. Accordingly, if the tumor increases to a size of 4 cm or more after the decided treatment with drug Y, the database watcher 290 determines the treatment result and notifies the manager agent 250.

Furthermore, the database watcher can be created with a predefined execution duration which defines a “life time” of the database watcher. Assume that in the given example the tumor size is supposed to start decreasing within 8 to 12 weeks after the treatment with drug Y. Accordingly, the life time of the database watcher can be determined to be 12 weeks. In other words, at the latest, after 12 weeks after the decided treatment, the effectiveness of drug Y for treatment of the given patient's brain tumor can be determined on the basis of the treatment result. This enables the doctor to verify whether his/her decided treatment is effective for the given patient. Alternatively, the execution duration of the database watcher can be unlimited so that the treatment result is only determined when the patient's medical record is closed, i.e., when the given patient is considered to be fully recovered or when the patient dies.

In the given example, the database watcher can be implemented by a periodic query against an underlying analysis data database (e.g., analysis data database 260 of FIG. 2). An exemplary query is shown in Table I below, which, for simplicity, is described in natural language without reference to a particular query language.

TABLE I
QUERY EXAMPLE

| | |
|---|---|
| 001 | FIND |

| | |
|---|---|
| 002 | Patient ID, Tumor Size |

| | |
|---|---|
| 003 | FROM |

| | |
|---|---|
| 004 | measurements |

| | |
|---|---|
| 005 | FOR |

| | |
|---|---|
| 006 | Patient ID = 123 |

Illustratively, the exemplary query shown in Table I is designed to retrieve tumor size values (lines 001-002) from a measurements database table (lines 003-004) for the given patient, who is illustratively identified by a patient identifier “123” (lines 005-008).

At step 390, the created database watcher is executed. An exemplary method for execution of a database watcher is described in more detail below with reference to FIG. 4. Method 300 then exits at step 392.

Referring now to FIG. 4, an exemplary method 400 for execution of a database watcher (e.g., database watcher 290 of FIG. 2) is illustrated. According to one aspect, method 400 is entered from step 390 of FIG. 3B. At least a portion of the steps of method 400 is performed using the expert system 220 and/or the intelligence unit 230 of FIG. 2.

Method 400 starts at step 410, where it is determined whether the execution duration of the database watcher has expired. If the execution duration is expired, processing proceeds from step 410 to step 430. Otherwise, it is determined at step 420 whether the trigger condition has occurred, i.e., whether the tumor size is 4 cm or more. If the trigger condition has not occurred, processing returns to step 410. Otherwise, processing proceeds from step 420 to step 430.

At step 430, a corresponding treatment result is determined for the given patient. In the given example, a current tumor size of the patient's brain tumor is retrieved from the measurements table in the underlying analysis data database. At step 440, at least one of the doctor, the underlying expert system, a quality control system, or the medical institution where the given patient is treated is notified with respect to the treatment result.

At step 450, a corresponding historical data database (e.g., historical data database 270 of FIG. 2) is updated with respect to the diagnosed disease, the recommended treatment, the decided treatment and the treatment result. Thus, feedback with respect to the patient's case is generated and can be used for future diagnosis and treatment decisions. Specifically, the feedback can be used to update the underlying expert system. Processing then continues at step 392 of FIG. 3B.

### CONCLUSION

In various embodiments, the invention provides numerous advantages over the prior art. For instance, according to embodiments of the invention an outcome of a decided treatment can be reported with added confidence/weight when enough information has been gathered. Such intelligence is built as historical data used to draw valuable conclusions for later diagnoses. With the use of statistical data and probability, more related probability and statistics can be produced. For instance, information such as a frequency of how often a particular doctor overrides recommended treatments as well as his/her success and failure rates can be captured and calculated into probability of patient treatment for a specific doctor's treatment based on statistical information as compared against doctor discretion. Accordingly, the intelligence of the expert system can be built based on historic, statistical data based on previous treatments by various doctors for a specific case and also based on probabilities of success rates determined for a specific doctor's treatment methods. Such historical data can be used to track the progress or likelihood of errors committed based on the decided treatment by a doctor. For instance, if a doctor disagrees with the system 25% more often than the average and he/she has a success rate in similar cases which is 10% below average, such intelligence can be captured and built into the expert system.

While the foregoing is directed to embodiments of the present invention, other and further embodiments of the invention may be devised without departing from the basic scope thereof, and the scope thereof is determined by the claims that follow.

What is claimed is:

1. A computer-implemented method of evaluating treatment decisions, comprising:
receiving a specification of a decided treatment for a patient having a given disease, the decided treatment differing from a recommended treatment identified by an expert system in response to analysis of symptomatic data corresponding to the patient;
creating an outcome test specification defining at least one check point with respect to the given disease which is suitable for evaluation of a treatment result obtained with the decided treatment;
creating, on the basis of the outcome test specification, a database watcher configured to analyze data which is subsequently provided for the patient with respect to the at least one check point to monitor progress of the given disease;
executing the database watcher to determine the treatment result on the basis of the analyzed data; and
notifying a medical institution of the determined treatment result.

2. The method of claim 1, further comprising:
comparing the decided treatment and the recommended treatment to identify all deviations of the decided treatment from the recommended treatment.

3. The method of claim 1, wherein the at least one check point identifies one of a diagnosis and a test to be performed on the patient to provide feedback to the database watcher with respect to the progress of the given disease.

4. The method of claim 1, further comprising at least one of:
(i) auditing a doctor having determined the decided treatment to identify reasons for overriding the recommended treatment; and
(ii) notifying the medical institution of the decided treatment.

5. The method of claim 1, further comprising:
creating, for the at least one check point, a trigger condition to trigger determination of the treatment result, wherein the treatment result is determined only if the trigger condition is satisfied.

6. The method of claim 1, further comprising:
associating the database watcher with a predefined execution duration.

7. The method of claim 1, further comprising:
comparing the treatment result with a result which has previously been obtained for another patient by adopting the recommended treatment; and
updating the expert system on the basis of the comparison.

8. The method of claim 1, further comprising:
persistently storing the symptomatic data, the decided treatment and the treatment result.

9. A computer-implemented method of evaluating treatment decisions, comprising:
receiving a specification of a decided treatment for a patient having a given disease, the decided treatment differing from a recommended treatment identified by an expert system in response to analysis of symptomatic data corresponding to the patient;
monitoring the patient subsequent to being treated according to the decided treatment;
evaluating the decided treatment on the basis of data captured by the monitoring; and
on the basis of the evaluation, generating feedback with respect to the decided treatment and the recommended treatment.

10. The method of claim 9, wherein monitoring the patient comprises:
creating an outcome test specification defining at least one check point with respect to the given disease which is suitable for evaluation of a treatment result obtained with the decided treatment;
creating, on the basis of the outcome test specification, a database watcher configured to analyze the captured data which is subsequently provided for the patient with respect to the at least one check point to monitor progress of the given disease; and
executing the database watcher to determine the treatment result on the basis of the analyzed data.

11. The method of claim 10, wherein the at least one check point identifies one of a diagnosis and a test to be performed on the patient to provide feedback to the database watcher with respect to the progress of the given disease.

12. The method of claim 10, further comprising:
creating, for the at least one check point, a trigger condition to trigger determination of the treatment result, wherein the treatment result is determined only if the trigger condition is satisfied.

13. The method of claim 10, further comprising:
associating the database watcher with a predefined execution duration.

14. The method of claim 9, further comprising:
comparing the decided treatment and the recommended treatment to identify all deviations of the decided treatment from the recommended treatment, wherein the feedback includes all identified deviations.

15. The method of claim 9, further comprising at least one of:
(i) auditing a doctor having determined the decided treatment to identify reasons for overriding the recommended treatment; and
(ii) notifying a medical institution of the decided treatment.

16. The method of claim 9, wherein the feedback includes a conclusion regarding a doctor's judgment in electing the decided treatment instead of the recommended treatment.

17. The method of claim 9, further comprising:
comparing the treatment result with a result which has previously been obtained for another patient by adopting the recommended treatment, wherein the feedback includes a result of the comparison.

18. The method of claim 9, further comprising:
updating the expert system on the basis of the feedback.

19. The method of claim 9, further comprising providing the feedback to a medical institution.

20. A system, comprising:
an expert system configured to determine a recommended treatment in response to analysis of symptomatic data corresponding to a patient having a given disease; and
an intelligence unit for evaluating treatment decisions, the intelligence unit being configured to:
receive a specification of a decided treatment for the patient, the decided treatment differing from the recommended treatment;
create an outcome test specification defining at least one check point with respect to the given disease which is suitable for evaluation of a treatment result obtained with the decided treatment;
create, on the basis of the outcome test specification, a database watcher configured to analyze data which is subsequently provided for the patient with respect to the at least one check point to monitor progress of the given disease;
execute the database watcher to determine the treatment result on the basis of the analyzed data;
evaluate the decided treatment on the basis of the treatment result; and
generate, on the basis of the evaluation, feedback with respect to the decided treatment and the recommended treatment.
